(19) United States
(12) Patent Application Publication
(10) Pub. No.: US 2022/0133780 A1
(43) Pub. Date: May 5, 2022

(54) COMPOSITIONS AND METHODS FOR INTRACELLULAR IRON DISPLACEMENT PROTEINS

(71) Applicant: RUSS IP HOLDING GROUP LLC, Houston, TX (US)

(72) Inventor: Kenneth O. Russell, Houston, TX (US)

(21) Appl. No.: 17/573,235

(22) Filed: Jan. 11, 2022

Related U.S. Application Data
(63) Continuation of application No. 16/442,512, filed on Jun. 16, 2019, now abandoned.

(51) Int. Cl. A61K 33/24; A61K 33/06

(52) U.S. Cl. CPC A61K 33/24; A61K 9/0014; A61K 33/06

(57) ABSTRACT

Methods and pharmaceutical compositions for optimizing intracellular iron by displacing iron bound transferrin (Tf) protein on a mammalian cell surface prior to its binding to a transferrin receptor (TfR), in the delivery of chromium chloride as being a Tf-binding agent to displace iron bound, and in the treatment of conditions involving disturbances in iron metabolism.

### CROSS-REFERENCE TO RELATED APPLICATION

This is a Continuation application of application Ser. No. 16/442,512, filed on Jun. 16, 2019, which is a Continuation in-part of application Ser. No. 13/385,340, filed on Feb. 14, 2012, which is a continuation in-part of application Ser. No. 12/069,505, filed on Feb. 11, 2008, now U.S. Pat. No. 9,585,898, continuation in-part of application Ser. No. 15/925,437, filed on Mar. 19, 2018. The disclosure of which is hereby incorporated by reference herein in its entirety.

### FIELD OF INVENTION

The invention is based on the discovery that chromium in microvessels can displace iron on transferrin protein before it is pulled into the cell with chromium and iron via transferrin receptor.

The present invention relates to modification at the molecular level of an alternative pathway to receptor-mediated endocytosis (RME) of the Tf protein bound iron therein. The present invention has identified chromium chloride as being Tf-binding agent to displace iron bound to the Tf protein on a mammalian cell surface prior to its binding to the TfR. The present invention has convincingly shown that the chromium chloride can displace iron on Tf before it uptakes to a mammalian cell, therefore, identified as the first membrane-transferrin metallic iron displacement that prevents the mammalian cell from being iron overloaded and increase the ferroportin mediated efflux of iron out of the mammalian cell.

### BACKGROUND

Iron is essential for cell growth, proliferation and differentiation. It is also a necessary cofactor for the synthesis of neurotransmitters, dopamine, norepinephrine, and serotonin, and disruption of iron homeostasis may be involved in Parkinson's disease and/or mood disorders (Youdim, 1990).

Iron is required by most organisms as it serves as a prosthetic group for proteins involved in central cellular processes, including respiration, DNA synthesis and oxygen transport. In excess, cellular iron catalyzes the generation of free radicals that damage protein, DNA and lipids, whereas cellular iron deficiency impairs cellular proliferation. In humans, with the inherited diseases hemochromatosis, excess cellular iron can result in cirrhosis, cardiomyopathy and diabetes mellitus.

Maintenance of cellular iron homeostasis is accomplished by the coordinated regulation of iron uptake, storage and export by iron-regulatory proteins 1 and 2 (IRP1 and IRP2, also known as ACO1 and IREB2). Excess iron content in the brain is associated with several inherited neurodegenerative diseases, including neurodegeneration with brain iron accumulation (NBIA) and Friedreich's ataxia (FA), as well as common neurodegenerative disorders such as Parkinson's and Alzheimer's diseases. On the other hand, iron deficiency affects millions of people worldwide, and results in cognitive defects in children and anemia in adults. Cellular iron content must therefore be maintained and balanced within a narrow range to avoid the adverse consequences of iron deficiency or excess.

Iron related Reactive Oxygen Species (ROS) is responsible for early neuronal cell death. It is now established that oxidative stress is one of the earliest, if not the earliest, change that occurs in the pathogenesis of Alzheimer's disease (AD).

Mild cognitive impairment (MCI), a clinical precursor of AD, is characterized by elevations in oxidative stress. Increased iron was found at the highest levels both in the cortex and cerebellum from the pre-clinical AD/MCI cases. Interestingly, glial accumulations of redox-active iron in the cerebellum were also evident in preclinical AD patients and tend to increase as patients became progressively cognitively impaired. Research findings suggest that an imbalance in iron homeostasis is a precursor to the neurodegenerative processes leading to AD and is not necessarily unique to affected regions.

Iron accumulates as a function of age in several tissues in vivo and is associated with the pathology of numerous age-related diseases. The molecular basis of this change may be due to a loss of iron homeostasis at the cellular level. Total iron content increases exponentially during cellular senescence. Iron accumulation occurs during normal cellular senescence in vitro. This accumulation of iron may contribute to the increased oxidative stress and cellular dysfunction seen in senescent cells. (David W. Killilea, Hani Atamna, Charles Liao, Bruce N. Ames. Antioxidants & Redox Signaling. October 2003, 5(5): 507-516).

A recent study examined the effects of iron overload on telomere length and telomerase activity. Mean telomere lengths were similar in iron-loaded and control livers. However, telomerase activity was increased 3-fold by iron loading. Telomeres are repeated sequences (TTAGGG) at the ends of chromosomes that are incompletely copied when DNA is replicated during mitosis.

Iron overload is also common and equally detrimental, affecting parenchymal organs including the liver, heart, and pancreas. In Western populations iron overload is mostly genetic due to hereditary hemochromatosis (HH), caused by mutations in genes involved in the sensing of systemic iron levels (such as HFE, HJV, and TFR2), or to disorders that cause ineffective erythropoiesis and secondary iron loading (e.g., thalassemias). There is increasing awareness that acquired metabolic disorders can also cause iron overload, which may exacerbate pathogenesis (Pietrangelo, 2016).

In cells that lack a mechanism to restore telomeric sequences, telomeres shorten progressively with each round of cell division. When telomeres reach a threshold length, cells withdraw from the cell cycle and acquire a senescent phenotype.

Thus, the inexorable shortening of telomeres with each round of cell division is regarded as a “mitotic clock” that records the number of antecedent cell divisions and signals the onset of phenotypic alterations associated with aging. A substantial body of data indicates that telomere attrition is modulated by oxidant-antioxidant balance.

The finding that telomere lengths were not dramatically altered by iron loading suggested that telomerase activity might be increased in the iron loaded livers. (Kyle E. Brown, et al, Increased hepatic telomerase activity in a rat model of iron overload: a role for altered thiol redox state, Free Radic Biol Med. 2007 Jan. 15; 42(2): 228-235).

Iron deficiency is the most common cause of anemia and represents a global health problem. Iron-deficiency anemia is defined by low numbers of small (microcytic) and hypoferremic erythrocytes. Iron deficiency may contribute to cognitive developmental defects in children, poor physical performance, and unfavorable pregnancy outcomes (Camaschella, 2015).

Iron deficiency in children results in auditory defects from disruption of myelin (Roncagliolo et al., 1998), and demylinating diseases such as multiple sclerosis are associated with defects in cellular iron homeostasis (Drayer et al., 1987).

Ferric iron is carried in the bloodstream in association with a transferrin protein (Baker and Morgan, 1994). Transferrin protein with iron is endocytosed into cells following binding to the cell surface transferrin receptor. Transferrin may carry iron from the RPE to the photoreceptors via a Tf-TfR-dependent mechanism (Yefimova et al., 2000).

Recent studies suggest that abnormal retinal iron metabolism may promote a variety of retinal disorders. These include ocular sideros is either from intraocular foreign bodies or from intraocular hemorrhage.

Retinal degeneration has also been observed in hereditary disorders resulting in iron overload, including aceruloplasminemia, hereditary hemochromatosis, pantothenate kinase associated neurodegeneration (formerly Hallervorden-Spatz Disease), and Friedreich's Ataxia.

Recently, evidence suggests that iron overload may also play a role in the pathogenesis of age-related macular degeneration (AMD) that leads to vision loss. Possible mechanisms of this vision loss include direct iron toxicity to the photoreceptors, iron toxicity or mechanical damage to the RPE, cellular migration and proliferation in the subretinal space, proliferation of fibrovascular membrane, or separation of photoreceptors from the RPE (Gillies and Lahav, 1983).

Recent researches suggest that iron regulation may play a role in the treatment of a number of neurological diseases such as Alzheimer's disease and Parkinson's disease, Huntington's disease and Friedreich's Ataxia (Zheng et al., 2005; Richardson, 2004). It is plausible that iron chelation may also be useful in retinal disease associated with iron overload.

Until recently, the only iron chelator in widespread clinical use in the United States was deferoxamine B (DFO), and despite being a relatively effective iron chelator for the treatment of transfusional iron overload, it has many notable limitations. The drug is an inefficient iron chelator, as only 5% or less of the drug administered promotes iron excretion (Bergeron et al., 2002).

In addition, because the iron chelator is poorly absorbed by the gastrointestinal system, and its elimination from the body is rapid, effective DFO treatment requires subcutaneous (SC) or IV administration for 9 to 12 hours for 5 or 6 days each week (Lee et al., 1993; Pippard, 1989). Therefore, for chronic treatment, chelation with DFO is costly, inefficient, cumbersome, and unpleasant.

In addition, DFO administration can have some rare but potentially serious side effects, including pulmonary toxicity, bony changes, growth failure, and promotion of Yersinia enterocolitica infections (Tenenbein et al., 1992; Brill et al., 1991; De Virgiliis et al., 1988).

Other iron chelators have been put into clinical use, including deferiprone (L1) and deferasirox (Exjade). Deferiprone has the advantage of being orally active and has been shown to be a more efficient iron chelator than DFO in removing cardiac iron, the cause of most of the mortality in transfusional iron overload (Anderson et al., 2002). A recent report demonstrates the ability of L1 to decrease brain iron in patients with Friedreich's Ataxia (Boddaert et al., 2007). This result suggests that L1 may similarly decrease retinal iron levels.

Deferiprone has rare but serious side-effects, including hepatic fibrosis, agranulocytosis, neutropenia, and arthropathy (Olivieri et al., 1986; Cohen et al., 2003; Ceci et al., 2002). The cause of deferiprone-related side effects is not known, but it may be deferiprone is a bidentate iron chelator.

At low concentrations, bidentate iron chelators can facilitate the formation of free-radicals from the formation of incomplete iron chelator complexes (Hershko et al., 2005). Since three molecules of deferiprone are required to completely remove iron from the labile pool, low levels of deferiprone can leave iron incompletely chelated and may cause the unbound portion of iron to be an even more effective catalyst for the generation of free radicals.

Deferasirox is an iron chelator that has just been recently approved for clinical use in patients with iron overload due to blood transfusion. Deferasirox is orally active and has an extended half-life, allowing for once-daily oral dosing (Vanorden and Hagemann, 2006). Current data show deferasirox to be as effective an iron chelator as subcutaneous deferoxamine, which is the current drug of choice for chronic iron overload patients (Piga et al., 2002).

Another potentially therapeutic iron chelator with interesting properties is salicylaldehyde isonicotinyl hydrazone (SIH). This iron chelator can protect cultured cardiomyocytes from oxidative stress induced death at concentrations 1000 fold lower than DFO (Simunek et al., 2005). However, SIH has poor stability in an aqueous environment due to the rapid hydrolysis of its hydrazone bond.

There are many challenges with using these clinically-available iron chelators to prevent and treat retinal degeneration. Ideally, an iron chelator should be selectively bind iron, but not other biologically important divalent metals such as Zinc (Liu and Hider, 2002).

In addition, an effective iron chelator must reach its target sites at a sufficiently high level. The chelator must be able to be absorbed in sufficient quantity through the gastrointestinal tract, the blood-brain barrier, or in the case of the retina, the blood-retina barrier (BBB). Thus, to successfully penetrate the blood-brain/blood-retinal barrier, an iron chelator must possess appreciable lipid solubility (Kalinowski and Richardson, 2005) and small molecular size, ideally below 500 Daltons (Maxton et al., 1986).

Iron must be carefully regulated and optimized to provide necessary iron levels without causing oxidative damage in the photoreceptors, where there is a high oxygen tension and high concentration of easily oxidized polyunsaturated fatty acids,

Iron that is not utilized or stored by the cell may be exported by the transport protein ferroportin (also known as MTP-1 or IREG-1) (Donovan et al., 2000; Abboud and Haile, 2000; McKie et al., 2000). Iron is exported by ferroportin in its ferrous state and must be oxidized to be accepted by circulating transferrin.

The oxidation of ferrous iron is accomplished by ferroxidases, ceruloplasmin and hephaestin. Ceruloplasmin is a copper binding protein, which contains over 95% of copper found in plasma. Hephaestin has 50% homology to ceruloplasmin and has ferroxidase activity. Unlike ceruloplasmin, which is present as a secreted plasma protein and glycosylphosphatidylinositol (GPI)-anchored protein (Patel and David, 1997), hephaestin is present only as a membrane-bound protein.

The opposing requirements and toxicities of iron are managed by an iron-responsive mechanism of post-transcriptional regulation of key iron-handling proteins (Hentze and Kuhn, 1996). This regulation allows individual cells to regulate iron uptake, sequestration, and export according to their iron status.

Further, there is no known mechanism of iron excretion from the body. Roughly 1-2 mg of iron is lost daily through sweat, blood loss, sloughing of intestinal epithelial cells, and desquamation. To compensate for this loss, the body absorbs about 1-2 mg of dietary iron per day, but hemoglobin synthesis alone requires 20-25 mg of iron per day.

To support hemoglobin synthesis and other metabolic processes, iron must be recycled and tightly regulated within the system instead of chelation. The circulating peptide hormone hepcidin together with its receptor ferroportin primarily maintain systemic iron homeostasis, whereas iron-regulatory proteins play a primary role in the control of intracellular homeostasis (https://www.ncbi.nlm.nih.gov/pmc/articles/PMC4464783/).

In average, while the iron of 4˜5 g is contained but the human body exists among the enzyme, and hemoglobin and myoglobin iron is held in the form of the ferritin and haemosiderin as storage. It is combined within the hemoglobin of the red blood cell while such iron, approximately, the half, in other words, about 2 g exists as the heme iron. It has only the lifetime (75-150) in which such red blood cell is limited. Therefore new red blood cells have to be constantly formed and it has to be removed to old. By recirculating the iron obtained in this way for the metabolism of iron by the red blood cell in which the high reproduction capacity is aged being slaughtered and is eaten and dissolving these it is achieved. In this way, in this way, necessary demand of the iron of about 25 mg is mostly provided to the erythropoiesis.

The management of iron levels and delivery is also a major challenge. Human cells accumulate iron from two main circulating sources. The first one, which is a classical source, consists of iron bound to transferrin, as described below, and the second one is called Non-Transferrin-Bound Iron (NTBI).

Most cell use transferrin, a serum protein, as a primary staple iron source/transporter. Transferrin comprises a class of biological iron-binding proteins, each lobe bearing a single site capable of reversibly binding iron and accounting for the physiological roles of the proteins in iron transport and iron withholding as a defense against infection.

Transferrin (Tf) normally provides iron for cellular needs and for most cells, the delivery of transferrin-bound iron depends on association of the protein with transferrin receptors, TfR1 and TfR2, on plasma membranes. An elaborate receptor-mediated pathway drives endocytosis of Tf-bound iron into mammalian cells for use and storage. Thus, TfR1 and TfR2 play critical roles in iron transfer involving transferrin.

For iron deficient patients, an effective transport of iron from external sources into the cells is required. This requirement is complicated by the fact that environmental iron is invariably present as insoluble iron leading to poor bioavailability and toxicity. Therefore, activators which provide efficient uptake and transport systems to extract iron from their environment and ferritins that store iron in a non-toxic form are required.

Iron-regulatory proteins (IRPs) register intracellular iron status and, in cases of intracellular iron deficiency, bind to iron-responsive elements (IREs) on the mRNA of the regulated protein. Binding of IRPs to the IRE of ferritin sterically obstructs efficient translation, which decreases ferritin levels in iron-deficiency. In contrast, binding of IRP to the IRE of transferrin receptor protects mRNA from degradation, which increases transferrin receptor in iron-deficiency.

An alternate theory of Alzheimer's Disease and diabetes holds that Alzheimer's Disease is not caused by the increase in insulin from Type II diabetes. Rather, there is improper insulin handling occurring directly in the brain (probably from inadequate chromium).

People are calling this Type III Diabetes. Previous studies have suggested an acutely improving effect of insulin on memory function. Subjects after insulin reported signs of enhanced mood, such as reduced anger and enhanced self-confidence. Results indicate a direct action of prolonged intranasal administration of insulin on brain functions, improving memory and mood in the absence of systemic side effects. (Benedict C, et al, Intranasal insulin improves memory in humans, Psycho neuroendocrinology. 2004 November; 29(10):1326-34).

Conditions in which glucose metabolism is impaired due to insulin resistance are associated with memory impairment. It was hypothesized that supplemental chromium (Cr) may alleviate insulin resistance in type 2 diabetes and consequently improve memory acquisition, depending upon its source and level. High-fat diet caused a 32% reduction in expressions of glucose transporters 1 and 3 (GLUTs) in brain tissue and a 27% reduction in mean percentage time spent in the target quadrant and a 38% increase in spatial memory acquisition phase (SMAP) compared with ND. Compared with supplemental Cr as CrAc, CrGly was more effective to ameliorate response variables (i.e., restoration of tissue Cr concentration, enhancement of cerebral GLUTs expressions, and reduction of the glucose/insulin ratio and SMAP) in a dose-response manner, especially in rats fed HFD.

Supplemental Cr as CrGly may have therapeutic potential to enhance insulin action and alleviate memory acquisition in a dose dependent manner, through restoring tissue Cr reserve and enhancing cerebral GLUTs expressions. (Sahin K, et al, The Effects of Chromium Complex and Level on Glucose Metabolism and Memory Acquisition in Rats Fed High-Fat Diet. Bio! Trace Element Res. 2010 Dec. 1)

Chromium helps to promote conversion of tryptophan to serotonin by facilitating absorption into muscle tissue of the amino acids that compete with tryptophan for access to the brain.

Iron circulating in transferrin in the blood cannot directly cross the blood brain barrier (BBB). There are several pathways that can transfer iron across the BBB. The first and probably most common is through transferrin receptors on brain endothelial cells, which bind iron circulating in the form of transferrin. The transferrin receptor or bound complex then enters the brain by endocytosis. Several other transporter systems may also deliver iron across the BBB, such as the divalent metal transporter and the lactoferrin receptor.

In addition, these pathways, especially the transferrin-receptor mediated pathway, are the main avenues for iron transport within the CNS (i.e., into various cell types of the brain). The amount of iron taken up and stored by the cells is a function of the abundance of the transferrin receptor and its ligand. Ferritin is the most common iron-storage protein in the brain. Another sequestrant of iron found in high concentrations in the substantia nigra and locus ceruleus is neuromelanin. There is evidence to suggest that neuromelan in acts to reduce potentially toxic iron by chelating iron found in the cytosol of neurons. Finally, after the brain uses the iron it has stored, the iron must leave the cell, and the copper-associated protein ceruloplasmin may facilitate cellular release of iron.

Transferrin proteins exist for the purpose of transporting irons from the gut to the various parts of the body for uptake into the cells by transferrin receptors on cell membranes. Irons might otherwise be subject to chelation by glucose and routinely excreted in the urine.

When iron and chromium from the diet are absorbed into the enterocytes of the gut there are perhaps 2000 times as many iron atoms competing for the same di-metal transport receptors that pull iron or chromium atoms into the enterocytes of the duodenum. Chromium absorption is therefore highly numerically disadvantaged. The typical healthy modern human absorbs only about 10% of the iron ingested while only 0.4% of chromium chloride is absorbed from the contents of the gut. This numerical advantage for iron is further advanced in the competition for safe sequestration on the transferrin proteins in the blood at the gut (after exiting the enterocytes). Iron or chromium atoms that are not safely sequestered on transferrin proteins (in the blood at the gut) are subject to being chelated by glucose in the blood. Iron gluconate and chromium gluconate are subject to be excreted in the urine by the kidneys.

The advantage of transdermal chromium delivery (as opposed to oral or intravenous delivery) is that chromium is introduced into the micro-vessels of the venous return blood system below/within the skin. Transferrin proteins have traversed every other body tissue with ample opportunity to download iron. This means that the highest concentration of apotransferrin proteins (without iron on board) are about to begin the transit back to the gut and liver for the purpose of picking up a new supply of iron or chromium from the gut.

Chromium can displace iron on the transferrin proteins with introducing chromium into micro-vessels of the venus return system (with an abundance of apo-transferrin proteins) will insure that more chromium atoms are safely sequestered on apo-transferrin proteins and ultimately endocytosed into cells to optimize insulin signaling. This also reduces iron uptake into the cells by the displacement of iron by chromium on the apo-transferrin proteins.

Thus, there is a need for effective intracellular iron regulatory mechanism that maintains cellular iron homeostasis within a narrow range to avoid the adverse consequences of iron deficiency or excess.

### Definitions

The terminology used herein is for the purpose of describing particular embodiments only and is not intended to be limiting. As used in the specification and the appended claims, the singular forms “a,” “and” and “the” include plural references unless the context clearly dictates otherwise.

“About” as used herein may refer to approximately a +/−10% variation from the stated value. It is to be understood that such a variation is always included in any given value provided herein, whether specific reference is made to it.

“Displacement” as used herein refers to transferrin-bound iron ion to be displaced or iron ion is inhibited to bind the transferrin.

“Binding Protein” is used herein to refer to a monomeric or multimeric protein that binds to and forms a complex with a binding partner, such as, for example, a polypeptide, an antigen, a chemical compound or other molecule, or a substrate of any kind. A binding protein specifically binds a binding partner.

“Pharmaceutically acceptable medium” means a medium that is compatible with the skin, mucous membranes and the integuments.

A “penetration enhancer” is an agent known to accelerate the delivery of the drug through the skin. These agents also have been referred to as accelerants, adjuvants, and absorption promoters, and are collectively referred to herein as “enhancers.”

The term “fatty acid” means a fatty acid that has four (4) to twenty-four (24) carbon atoms.

The term “supracutaneous administration” means transdermal administration, topical administration, or any combination of the composition

The term “optimizing” to be understood to include balancing, reducing, inhibiting, treating, delaying, improving, and the like.

“Pharmacologically effective amount” means that the concentration of the drug is such that in the composition it results in a therapeutic level of drug delivered over the term that the gel is to be used. Such delivery is dependent on a number of variables including the drug, the form of drug, the time period for which the individual dosage unit is to be used, the flux rate of the drug from the gel, surface area of application site, etc. The amount of drug necessary can be experimentally determined based on the flux rate of the drug through the gel, and through the skin when used with and without enhancers.

“Fixed combination” should be understood as meaning a combination whose active principles are combined at fixed doses in the same vehicle/medium (single formula) that delivers them together to the point of application.

“Treatment” as used herein refers to any treatment of a human condition or disease and includes: (1) preventing the disease or condition from occurring in a subject which may be predisposed to the disease but has not yet been diagnosed as having it, (2) inhibiting the disease or condition, i.e., arresting its development, (3) relieving the disease or condition, i.e., causing regression of the condition, or (4) relieving the conditions caused by the disease, i.e., stopping the symptoms of the disease.

### SUMMARY OF THE INVENTION

The present invention provides an iron displacing agent, which can be provided in the form of a pharmaceutical composition that includes, optionally, a pharmaceutically acceptable carrier. The iron displacing agent can be used in a method of facilitating excessive iron transferring from a cell to bone marrow for Red Blood Cell (RBC) reproduction.

The method comprises contacting microvessels of a cell with transferrin-binding agent. Also provided is a method of treating a disorder associated with excess iron in cells in a subject. The method comprises administering to the subject an effective amount of the composition of the invention. The subject is typically a mammal, most typically a human or veterinary subject.

In one embodiment, the present invention is directed to a pharmaceutical composition for supracutaneous administration comprising at least one active pharmaceutical ingredient (e.g., CrCl₃.6H₂O (actually trans-[Cr(H₂O)₄Cl₂]Cl.2H₂O) or a synthetic transferrin binder. In a broad aspect of the invention, the active ingredients employed in the composition may include salt (e.g., magnesium sulfate (MgSO₄(H₂O)x)) for vasodilation to increase skin absorption rate.

In one embodiment, the composition comprises a penetration enhancing agent, a thickening agent, and water. Additionally, the present invention may optionally include emollients, stabilizers, antimicrobials, fragrances, and propellants.

The penetration enhancing agent has a function of improving the solubility and diffusibility of the drug, and those which improve percutaneous absorption by changing the ability of the stratum corneum to retain moisture, softening the skin, improving the skin's permeability, acting as penetration assistants or hair-follicle openers or changing the state of the skin such as the boundary layer.

Further, the penetration enhancing agent herein is a functional derivative of a fatty acid, which includes isosteric modifications of fatty acids or non-acidic derivatives of the carboxylic functional group of a fatty acid or isosteric modifications thereof.

Non-limiting examples of penetration enhancers include C8-C22 fatty acids such as isostearic acid, octanoic acid, and oleic acid; C8-C22 fatty alcohols such as oleyl alcohol and lauryl alcohol; lower alkyl esters of C8-C22 fatty acids such as ethyloleate, isopropyl myristate, butyl stearate, and methyllaurate; di(lower)alkylesters of C6-C8 diacids such as diisopropyl adipate; monoglycerides of C8-C22 fatty acids such as glyceryl monolaurate; tetrahydrofurfuryl alcohol polyethyleneglycol ether; polyethylene glycol, propylene glycol; 2-(2-ethoxyethoxy)ethanol; diethylene glycol monomethy]ether; alkylarylethers of polyethylene oxide; polyethyleneoxide monomethylethers; polyethylene oxide dimethyl ethers; dimethyl sulfoxide; glycerol; ethyl acetate; acetoacetic ester; N-alkylpyrrolidone; and terpenes.

The thickening agent or thickener used herein may include anionic polymers such as polyacrylic acid (CARBOPOL® by B.F. Goodrich Specialty Polymers and Chemicals Division of Cleveland, Ohio), carboxymethyl cellulose and the like. Additional thickeners, enhancers and adjuvants may generally be found in United States Pharmacopeia/National Formulary (2000); Remington's The Science and Practice of Pharmacy, Meade Publishing Co.

Because of high lipophilicity, the pharmaceutical composition can be administered transdermally as chromium chloride readily enter cells to displace excessive iron from holo-transferrin, therefore, is able to efficiently block the production of iron-catalyzed formation.

In one embodiment, the disorder associated with elevated iron levels in cells, which are found in the retinas of patients with age-related macular degeneration. This suggests that iron may play a role in the pathogenesis of neovascular blood vessels.

In one embodiment, the disorder associated with excess iron in cells is a chronic obstructive pulmonary disease (COPD) associated with increased iron deposition in lung tissues.

In one embodiment, the disorder associated with excess iron in cells is the anemia of inflammation. Examples of anemia associated with inflammation (or inflammatory block) include rheumatoid arthritis and anemia that is secondary to infection. In these circumstances, iron accumulates in macrophage cells and cannot be recycled to developing red cells.

In one embodiment, the disorder associated with excess iron content in the brain is associated with inherited neurodegenerative diseases, including neurodegeneration with brain iron accumulation (NBIA) and Friedreich's ataxia (FA), as well as common neurodegenerative disorders such as Parkinson's and Alzheimer's diseases.

In one embodiment, the disorder associated with iron deficiency affects cognitive defects in children and anemia in adults.

In one embodiment, macrophages digest senescent red cells and then recycle the iron that is contained within the hemoglobin molecules by exporting inorganic iron (via ferroportin). This invention has shown that, in addition, iron on transferrin protein can be displaced by chromium element before it is pulled into a cell via transferrin protein receptor. The exit of inorganic iron is blocked when there is inflammation. Anemia occurs because developing red cells have insufficient iron. Macrophage recycling red cells could be facilitated by maximizing the exit of iron using the method of this invention.

In one embodiment, the disorder is related to systemic iron overload, such as occurs with hemochromatosis or related iron disorders. Related iron disorders include iron-overload from transfusion or chronic hemolysis. In this type of disorder, the method of this invention can be used to facilitate transferring excessive iron from cells to the bone marrow to reproduce red cells the body.

In one embodiment, the present invention can also be used in disorders where the excess of iron in cells leads to cell-specific toxicities. These include cellular iron stress due to iron overload in cells and oxidative stress. Because the iron is highly expressed in tissues such as brain and kidney, the method of the invention can be used to reduce tissue damage in these areas that is caused by such stress.

Although the invention has been described with respect to specific embodiments and examples, it should be appreciated that other embodiments utilizing the concept of the present invention are possible without departing from the scope of the invention. The present invention is defined by the claimed elements, and any modifications, variations, or equivalents that fall within the true spirit and scope of the underlying principles.

### BRIEF DESCRIPTION OF FIGURES

FIG. 1 shows chromium atom displaces iron bound on the transferrin (Tf1) protein prior to its pulling to cytoplasm of the cell membrane via transferrin receptor (TfR1). Chromium binding insulin receptor (IR) activates the signaling TfR2 to pull iron atom from TfR3 only one therefore, regulates and optimizes intracellular iron metabolism.

Chromium displacement of iron on the Tf protein occurs most efficiently in the venus return microvessels of the skin. The activated IR signals for the translocation of the GLUT4 receptor from the vesicles of the cytosol to the surface of the cell membrane to increase glucose uptake.

The GLUT4 receptor opens a pore in the cell membrane and pulls glucose through the pore via passive diffusion. The TfR1 protrudes through the cell membrane and captures a Tf1 protein from the blood, which is then endocytosed (pulled through the cell membrane into the cell) with chromium and/or iron on the Tf1 protein.

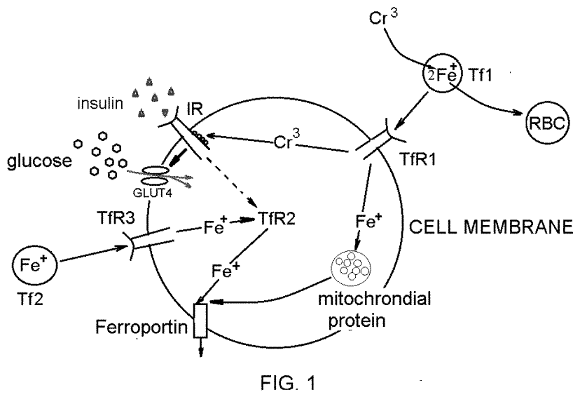

Still with FIG. 1, chromium translocation begins on the IR for installation on four available slots on the chromodulin component of the insulin intracellular subunit. With chromium installed on the chromodulin component, the IR signaling event is not subject to repetitive interruption/deactivation (which reduces that rate of glucose uptake by approximately 85% and increases the period of hyperglycemia by about 600%).

With chromium absent the chromodulin component, the IR, the TfR1, and the GLUT4 receptor will necessarily be reactivated approximately six times during the period of hyperglycemia. Six activations of the TfR1 may result in six iron loading events within the cell. With chromium installed chromodulin component, only one TfR1 iron loading event will occur. When the feeding event is concluded and hypoglycemia is achieved the chromium-chromodulin component is ejected from the cell and the chromium is believed to be lost via the urine.

The compositions are administered in any suitable manner, often with pharmaceutically acceptable carriers. Suitable methods of administering compositions in the context of the present invention to a subject are available, and, although more than one route can be used to administer a particular composition, a particular route can often provide a more immediate and more effective reaction than another route.

### METHODS AND USES OF THE INVENTION

The invention provides a method of facilitating heme-iron export from a cell. The method comprises contacting a cell with a high affinity heme-binding agent. Also provided is a method of treating a disorder associated with excess iron in cells in a subject. The method comprises administering to the subject an effective amount of the composition of the invention.

The subject is typically a mammal, most typically a human or veterinary subject. Examples of mammalian subjects include, but are not limited to, feline, canine, equine, porcine, bovine, ovine, primate, and rodent subjects.

In one embodiment, the disorder associated with excess iron in cells is the anemia associated with inflammation. Examples of anemia associated with inflammation (or inflammatory block) include rheumatoid arthritis and anemia that is secondary to infection. In these circumstances, iron accumulates in macrophage cells and cannot be recycled to developing red cells. Usually, macrophages digest senescent red cells and then recycle the iron that is contained within the hemoglobin molecules by exporting inorganic iron (via ferroportin) and heme-iron (via FLVCR). The exit of inorganic iron is blocked when there is inflammation.

Anemia occurs because developing red cells have insufficient iron. Macrophage recycling could be facilitated by maximizing the exit of heme-iron using the method of this invention.

In another embodiment, method is directed at certain other types of anemia, such as that which occurs with myelodysplasia, the macrocytic anemias, and anemias related to ineffective erythropoiesis. These latter anemias result from an imbalance of heme relative to globin. It appears that free heme is toxic to the developing red cell. By facilitating the export of excess heme, the method of the invention can help reduce this imbalance and thus improve red cell production.

In another embodiment, the disorder is related to systemic iron overload, such as occurs with hemochromatosis or related disorders. Related disorders include iron-overload from transfusion or chronic hemolysis. In this type of disorder, the method of the invention can be used to facilitate export of excess heme-iron into the stool via bile and thus out of the body. The invention can also be used in disorders where the excess of iron in cells leads to cell-specific toxicities. These include cellular iron stress due to iron overload in cells and oxidative stress. Because the heme-transporter FLUCR is highly expressed in tissues such as brain and kidney, the method of the invention can be used to reduce tissue damage in these areas that is caused by such stress.

Administration and Dosages

The compositions are administered in any suitable manner, often with pharmaceutically acceptable carriers. Suitable methods of administering compositions in the context of the present invention to a subject are available, and, although more than one route can be used to administer a particular composition, a particular route can often provide a more immediate and more effective reaction than another route.

The dose administered to a patient, in the context of the present invention, should be sufficient to effect a beneficial therapeutic response in the patient over time, or to inhibit disease progression. Thus, the composition is administered to a subject in an amount sufficient to elicit an effective response to the specific antigens and/or to alleviate, reduce, cure or at least partially arrest symptoms and/or complications from the disease. An amount adequate to accomplish this is defined as a “therapeutically effective dose.”

One skilled in the art will appreciate that the constituents of this formulation may be varied in amounts yet continue to be within the spirit and scope of the present invention. For example, the composition may contain per 100 g of the composition about 0.002 to about 0.01 g of chromium chloride, and about 10.0 to about 25.0 g of magnesium sulfate.

Toxicity and therapeutic efficacy of the active ingredients can be determined by standard pharmaceutical procedures, e.g., for determining LDS, (the dose lethal to 50% of the population) and the ED, (the dose therapeutically effective in 50% of the population). The dose ratio between toxic and therapeutic effects is the therapeutic index and it can be expressed as the ratio LD/ED. Compounds which exhibit large therapeutic indices are preferred. While compounds that exhibit toxic effects may be used, care should be taken to design a delivery system that targets such compounds to the site of affected tissue in order to minimize potential damage to uninfected cells and, thereby, reduce side effects.

Although the examples of the present invention involve the treatment of disorders associated with retina neurodegeneration related diseases, the composition and method of the present invention may be used to treat these disorders in humans and animals of any kind, such as dogs, pigs, sheep, horses, cows, cats, zoo animals, and other commercially bred farm animals.

The present invention is further illustrated by the following examples, which should not be construed as limiting in any way. The contents of all cited references throughout this application are hereby expressly incorporated by reference. The practice of the present invention will employ, unless otherwise indicated, conventional techniques of pharmacology and pharmaceutics, which are within the skill of the art.

Daily transdermal application of the composition significantly reduces the effect of diabetes on the neurovascular events of the retina and controls diabetes on the neurovascular changes under high serum glucose levels. Long-term treatment further decreases vascular endothelial growth factor (VEGF) expression.

In one embodiment, the present invention provides a composition containing effective amount of chromium chloride, magnesium sulfate, or any combination thereof to optimize and regulate intracellular, which leads to retinal degeneration related-diseases.

### EXAMPLES

The following examples are presented to illustrate the present invention and to assist one of ordinary skill in making and using the same. The examples are not intended in any way to otherwise limit the scope of the invention.

### Example 1

Iron Displacement on Transferrin Protein is Required Iron Homeostatis

Serum transferrin is a mammalian iron-transport protein. It has two specific metal-binding sites that bind a variety of metal ions in addition to ferric ion. Equilibrium constants for the binding of zinc(II) have been determined by difference UV titrations using nitrilotriacetic acid and triethylenetetramine as competing ligands. The values are log K1*=7.8 and log K2*=6.4 in 0.10 M N-(2-hydroxyethyl)piperazine-N′-2-ethanesulfonic acid and 15 mM bicarbonate, pH 7.4 at 25 degrees C. Titrations of the two forms of monoferric transferrin show that K1* corresponds to zinc binding to the C-terminal site and K2* corresponds to binding at the N-terminal site [1]. These results indicate that at serum bicarbonate concentrations, transferrin should have a higher affinity for zinc(II) than serum albumin and therefore could play some role in zinc transport. A linear free-energy relationship has been constructed which relates the formation constants of a series of zinc(II) and iron(II) complexes.

On the basis of the zinc-transferrin binding constants, this relationship has been used to estimate an iron (II)-transferrin binding constant of 10(7.4). Using this ferrous constant and literature values for the ferric transferrin binding constant, one calculates a ferric transferrin reduction potential of −140 mV, which is easily within the range of physiological reductants. Such as result tends to support a mechanism for iron displacement removal from the transferring, in which the ferric ion is reduced to the less tightly bound ferrous ion [2].

A two-step model of TfR mRNA regulation would also be compatible with previous data showing that iron chelation is not sufficient to induce TfR mRNA in quiescent T-cells, and a deletion of the 3′-regulatory region in TfR mRNA did not entirely abolish the growth-dependent continro alrrested [3].

### REFERENCES CITED IN EXAMPLE 1

[1] Harris W R, Thermodynamic binding constants of the zinc-human serum transferrin complex, 3920-6 (1983).
[2] M. W. Hentze, M. U. Muckenthaler, N. C. Andrews, Cell 117, 285 (2004).
[3] E. Nemeth et al., Science 306, 2090 (2004)

### Example 2

### Evidence of Iron Displacement Via the Transferrin-Binding Chromium Chloride

Transferrin (Tf)-bound two iron atoms (Tf-[Fe(III)]₂) binds to transferrin receptor (TfR1) on the cell surface where the Tf-[Fe(III)]₂-TfR1 complex is endocytosed. Acidification of the endosome causes the release of Fe(III) from Tf protein where it is reduced to Fe(II) by the STEAP3 oxidoreductase before export by DMT1 (divalent metal transporter 1). Apo-Tf/TfR1 complex is returned to the cell surface where it dissociates and initiates another round of iron uptake [1]. To gain insights into a structure and function of the Tf, we first studied the high affinity transferrin-binding of chromium chloride (FIG. 1).

Competitive binding of Fe(III), Cr(III), and Ni(II) to the Tf was investigated at various physiological iron to Tf protein concentration ratios. Loading percentages for these metal ions are based on a two M(n+) to one Tf (i.e., 100% loading) stoichiometry and were determined using a particle beam/hollow cathode-optical emission spectroscopy (PB/HC-OES) method.

Serum iron concentrations typically found in normal, iron-deficient from chronic disease, iron-deficient from inflammation, and iron-overload conditions were used to determine the effects of iron concentration on iron loading into Tf protein. The PB/HC-OES method allows the monitoring of metal ions in competition with Fe(III) for Tf protein binding.

Iron-overload concentrations impeded the ability of chromium (15.0 μM) or nickel (10.3 μM) to load completely into Tf. Low Fe(III) uptake by Tf under iron-deficient or chronic disease iron concentrations limited Ni(II) loading into Tf protein.

Competitive binding kinetic studies were performed in vitro with Fe(III), Cr(III), and Ni(II) to determine percentages of metal ion uptake into Tf as a function of time. The initial rates of Fe(III) loading increased in the presence of nickel or chromium, with maximal Fe(III) loading into Tf in all cases reaching approximately 24%. Addition of Cr(III) to 50% preloaded Fe(III)-Tf showed that excess chromium (15.0 μM) displaced roughly 13% of Fe(III) from Tf, resulting in 7.6±1.3% Cr(III) loading of Tf. The PB/HC-OES method provides the ability to monitor multiple metal ions competing for Tf binding and will help to understand metal competition for Tf binding [2].

The iron released from the Tf is exported into the circulation by ferroportin where it binds to apoTf for delivery to bone marrow for hemoglobin synthesis. We hypothesize that Tf-[Fe(III)]₂-TfR1 complex acts as a temporary docking site for Cr(III) after it binds to the Tf and prior to being picked up by insulin receptor (IR) in the circulation, and thus as a structural regulator of iron efficiency, or have another physiological functions.

### REFERENCES CITED IN EXAMPLE 2

[1] C. P. Anderson, et al., Biochimica et Biophysica Acta 1469 (2012).
[2] Quarles C D Jr., et al., J Biol Inorg Chem (2011).

Therefore, those skilled in the art will appreciate that the conceptions and specific embodiments disclosed in this description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

Throughout this application various publications are referenced. The disclosures of these publications in their entireties are hereby incorporated by reference into this application in order to describe more fully the state of the art to which this invention pertains.

As various modifications could be made to the exemplary embodiments, as described above with reference to the corresponding illustrations, without departing from the scope of the invention, it is intended that all matter contained in the foregoing description and shown in the accompanying drawings shall be interpreted as illustrative rather than limiting. Thus, the breadth and scope of the present invention should not be limited by any of the above described exemplary embodiments, but should be defined only in accordance with the following claims appended hereto and their equivalents.

We claim:

1. A method for regulating and optimizing intracellular iron metabolism by a displacement of iron-bound transferrin, the method comprising:
administering to microvessels of a mammalian cell an effective amount of composition,
wherein said composition comprises between about 0.002 to 0.01 g of chromium chloride in combination with between about 10 to 25 g of magnesium sulfate,
wherein the amounts of chromium and magnesium are selected together to provide a greater than additive effect.

2. The method of claim 1 is used for treating a disorder associated with excess or deficiency of iron in cells in a human subject.
